(19) United States
(12) Patent Application Publication
(10) Pub. No.: US 2019/0333606 A1
(43) Pub. Date: Oct. 31, 2019

(54) Early DNA Analysis Using Incomplete DNA Datasets

(71) Applicant: Technische Universiteit Delft, Delft (NL)

(72) Inventors: Zaid Al-Ars, Delft (NL); Ahmed Nauman, Delft (NL); Koenraad Laurent Maria Bertels, Delft (NL)

(73) Assignee: Technische Universiteit Delft, Delft (NL)

(21) Appl. No.: 16/348,171

(22) PCT Filed: Nov. 8, 2017
(86) PCT No.: PCT/NL2017/050721

§ 371 Date: May 8, 2019

(30) Foreign Application Priority Data
Nov. 9, 2016 (NL) 2017750

(51) Int. Cl. G16B 40/10; G16B 30/10; C12Q 1/6869; G16B 30/20; G16B 40/20; G16B 50/30

(52) U.S. Cl. CPC G16B 40/10; G16B 30/10; G16B 50/30; G16B 30/20; G16B 40/20; C12Q 1/6869

(57) ABSTRACT

A method of analyzing sequencing data associated with a sample is disclosed. The method comprises receiving by a computer system a plurality of sequencing reads while a sequencing assay is in progress of sequencing the sequencing reads, wherein at least some of the sequencing reads are incomplete sequencing reads, wherein the incomplete sequencing reads are sequencing reads for which only a part of the base pairs of the sequencing read have been determined by the sequencing assay, and the missing base pairs are still in the process of being determined by the sequencing assay. Further, mapping by the computer system the plurality of sequencing reads to a reference sequence. Further, predicting at least some of the missing base pairs and quality values of an incomplete sequencing read based on available base pair information of the sequencing reads and applying the predicted base pairs and quality values to the sequencing read.

### FIELD OF THE INVENTION

The present invention relates to analyzing sequencing data associated with a sample.

### BACKGROUND ART

DNA information can be used to diagnose genetic disease. The process of DNA data acquisition is a rather slow process, performed by DNA sequencers. This process may take days to complete. This long sequencing time, however, represents a bottleneck in using this data for many applications, such as (but not limited to) urgent diagnostics of genetic disease (such as aggressive cancer and neonatal patients), IVF screening, early identification of pathogens in the environment, ensuring food and water quality and safety, fast livestock DNA analysis, etc.

A method of analyzing sequencing data associated with a sample is known from US 2015/0066385. That method comprises receiving by a computer system a first sequencing read

associated with a sequencing assay while the sequencing assay is in progress, wherein the computer system comprises a processor, and comparing by the processor the first sequencing read
with another sequence to provide a comparison before the sequencing assay is complete.

### SUMMARY OF THE INVENTION

The present invention seeks to provide a rapid DNA analysis method.

According to an aspect of the invention, a method of analyzing sequencing data associated with a sample, the method comprising:

receiving by a computer system a plurality of sequencing reads while a sequencing assay is in progress of sequencing the sequencing reads, wherein at least some of the sequencing reads are incomplete sequencing reads, wherein the incomplete sequencing reads are sequencing reads for which only a part of the base pairs of the sequencing read have been determined by the sequencing assay, and the missing base pairs are still in the process of being determined by the sequencing assay;

mapping by the computer system the plurality of sequencing reads to a reference sequence; and

predicting at least some of the missing base pairs of an incomplete sequencing read and base pair quality values corresponding to the predicted base pairs, based on available base pair information of the sequencing reads and applying the predicted base pairs and the base pair quality values corresponding to the predicted base pairs to the incomplete sequencing read to obtain a completed sequencing read.

This has the advantage that the sequencing reads can be obtained as if the sequencing process were already completed. It allows many analyzing tools to be applied more quickly after sequencing has started. It may also enable the early transfer of data from one location to another. For example, to make the data accessible at multiple locations, and reducing the storage requirements at some locations. It may also enable taking early action based on the output of the analyzing tools, after sequencing has started but before it has completed.

The method may further comprise re-mapping the completed sequencing reads to the reference sequence. This improves the mapping of the sequencing reads.

The method may further comprise, upon receiving additional information about detected base pairs of the completed sequence from the sequencing assay, replacing by the computer system the predicted base pairs and the base pair quality values corresponding to the predicted base pairs of the completed sequence by the detected base pairs and base pair quality values corresponding to the detected base pairs. This enables the gradual improvement of the accuracy of the early action taken or analysis made based on the output of the analyzing tools.

The method may further comprise performing by the computer system variant calling, identifying a mutation in a DNA sample as compared to the reference DNA, or performing a DNA analysis algorithm by analyzing the completed sequencing reads.

The method may further comprise sequencing a plurality of short reads based on a sample using a sequencing assay; and streaming the plurality of short reads as they are being sampled from the sequencing assay to the computer system.

According to another aspect of the invention, a system for sequence analysis is provided comprising

a sequencing assay for sequencing a plurality of short reads based on a sample and streaming the plurality of short reads as they are being sampled from the sequencing assay to a computer; and

the computer system comprising:

a receiving unit for receiving a plurality of sequencing reads while the sequencing assay is in progress, wherein at least some of the sequencing reads are incomplete sequencing reads, wherein the incomplete sequencing reads are sequencing reads for which only a part of the base pairs of the sequencing read have been determined by the sequencing assay, and the missing base pairs are still in the process of being determined by the sequencing assay;

a mapping unit for mapping the plurality of sequencing reads to a reference sequence; and

a predicting unit for predicting at least some of the missing base pairs of an incomplete sequencing read based on available base pair information of the other sequencing reads and applying the predicted base pairs to the incomplete sequencing read to obtain a completed sequencing read.

The computer system may further comprise a re-mapping unit for re-mapping the complete sequencing reads to the reference sequence.

The computer system may further comprise an updating unit for, upon receiving additional information about detected base pairs of the completed sequence from the sequencing assay, replacing the predicted base pairs and the base pair quality values corresponding to the predicted base pairs of the completed sequence by the detected base pairs and base pair quality values corresponding to the detected base pairs.

The computer system may further comprise a calling unit for performing variant calling, identifying a mutation in a DNA sample as compared to the reference DNA, or performing a DNA analysis algorithm based on the completed sequencing reads.

According to another aspect of the present invention, a computer program product is provided comprising instructions for causing a computer system to perform a method set forth herein.

The person skilled in the art will understand that the features described above may be combined in any way deemed useful. Moreover, modifications and variations described in respect of the system may likewise be applied to the method and to the computer program product, and modifications and variations described in respect of the method may likewise be applied to the system and to the computer program product.

### BRIEF DESCRIPTION OF THE DRAWINGS

The present invention will be discussed in more detail below, with reference to the attached drawings.

FIG. 1 shows a flowchart of a method of DNA analysis.

FIG. 2 shows a flowchart of a method of completing incomplete DNA sequences.

FIG. 3 shows a flowchart of a method in which incomplete DNA sequences are streamed to a server and processed.

FIG. 4 shows a block diagram of a DNA sequencing apparatus including a sequencer and a server.

FIG. 5 shows an example of overlapping DNA short reads in relation to a reference genome.

### DETAILED DESCRIPTION

In the following, a system and method are described that can be used, for example, to perform DNA analysis on incomplete DNA datasets. This may allow to start the analysis early in the process, even before a DNA sequencers finish reading the DNA. This may be accomplished, for example, by connecting the DNA sequencer to a network and streaming the sequenced DNA information as soon as they are available to a compute server. Then, on the compute server, a number of algorithms are applied to overcome the limited data in the DNA and allow for identifying variants (i.e., mutations) in the incomplete sequenced DNA. The inventors were able to demonstrate the effectivity of the algorithms used to overcome the limited sequenced data in the DNA.

The invention may allow for a relatively quick identification of DNA variants, even before the acquisition of the DNA data has been completed. This may allow the early transfer of data from one location to another, once the data becomes available and before the sequencing is complete. This may also allow reducing the amount of time from taking a DNA sample, to being able to perform a diagnosis on DNA information. This may allow for new uses of DNA analysis that benefit from a very short turnaround time, such as IVF screening. It also may enable taking early action based on the output of the analyzing tools, after sequencing has started. For example, this may cover (but is not limited to):

Taking action on the source of the sample or its environment, such as diagnosis and treatment.

Taking action on the sample itself, such as using a different sampling procedure.

Taking action on the DNA sequencer, such as preemptive termination of a sequencing assay in progress, or freeing up the DNA sequencer to sequence a new sequencing assay.

Taking action on the computer system, such as the early termination of the computing process.

Taking action on the analyzing tools, such as introducing changes to the analyzing tools.

For example, the DNA analysis process starts by reading the DNA information by one or more sequencing machines. These machines read the DNA information and generate output in form of short sequences of characters (the characters may be called base pairs, while the sequences may be called short reads). These machines also assign a score to each base pair, referred to herein as a base-pair quality value, that represents the probability of incorrect base reading. These short reads typically range in size from tens to thousands of characters (typically a short read has 100 base pairs). However, the DNA is typically oversampled by the sequencing machines, generating more base pairs than the DNA has. In a particular example, the DNA is oversampled by a factor of 30; this is called 30× coverage. Generally, the oversampling factor may be larger than ten. Also, the sequencer may take a long time to complete its sequencing operation (1.5 days, for example).

According to the present disclosure, early analysis of DNA information may be performed before the sequencer is able to finish its operation. One complication in early analysis of DNA short sequences is that sequencers may generate all short reads base-by-base, in parallel. This means that first the first base pair of all short reads is generated, then the second base pair of all short reads is generated, and so on, until all reads are completed.

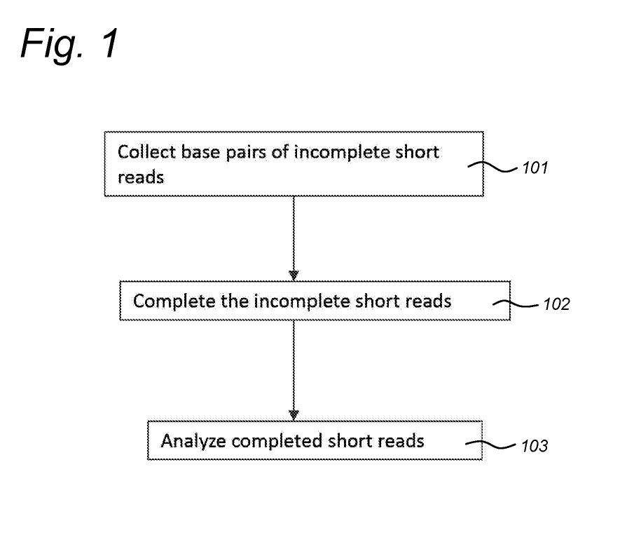

FIG. 1 shows a flowchart of an analysis method that can be performed on incomplete short reads. This method comprises step 101 of collecting base pairs and the corresponding base pair quality values of incomplete short reads, step 102 of completing the incomplete short reads, and step 103 of analyzing the completed short reads.

Step 101 may comprise connecting the sequencer to a computer, using for example a network connection or another kind of connection such as USB connection. Step 101 may comprise using the sequencer to process a DNA sample to generate the short reads. While processing the DNA sample, the sequencer may work to generate all short reads in parallel, wherein the sequencer constructs each short read by increasingly detecting more of the base pairs of the short reads. Thus, at any time during the sequencing, the sequencer has detected for each of the short reads a subset of all base pairs of that short read. The short reads are at that time thus incomplete in that only a portion of the base pairs of the short reads has been determined. Step 101 may further comprise sending (streaming) the incomplete short reads to the computer using the connection, and receiving the incomplete short reads by the computer. In an alternative embodiment, the sequencer and the computer are implemented as an integrated device that can perform both the functionalities of sequencing and analyzing.

Step 102 may involve using the oversampling of the DNA to reconstruct the short reads. Since each base pair of the genome is covered typically by a plurality of short reads, it is possible to use consensus information from other short reads that have already covered the missing base pairs to complete the short reads that are still incomplete. This way it is possible to trade-off the amount of oversampling with early analysis time. In other words, the information of several incomplete short reads and, if available, complete short reads, is combined. For example, the information that is missing in a first short read is obtained from a second short read. The base pair information obtained from the second short read is then used as base pair information of the first short read. When a plurality of short reads contain information about a particular base pair, a statistical method may be applied to determine the final value of that particular base pair to complete the short read. Similarly, a base pair quality value of the missing bases can be determined using the base pair quality values of the reads and applying an appropriate statistical method.

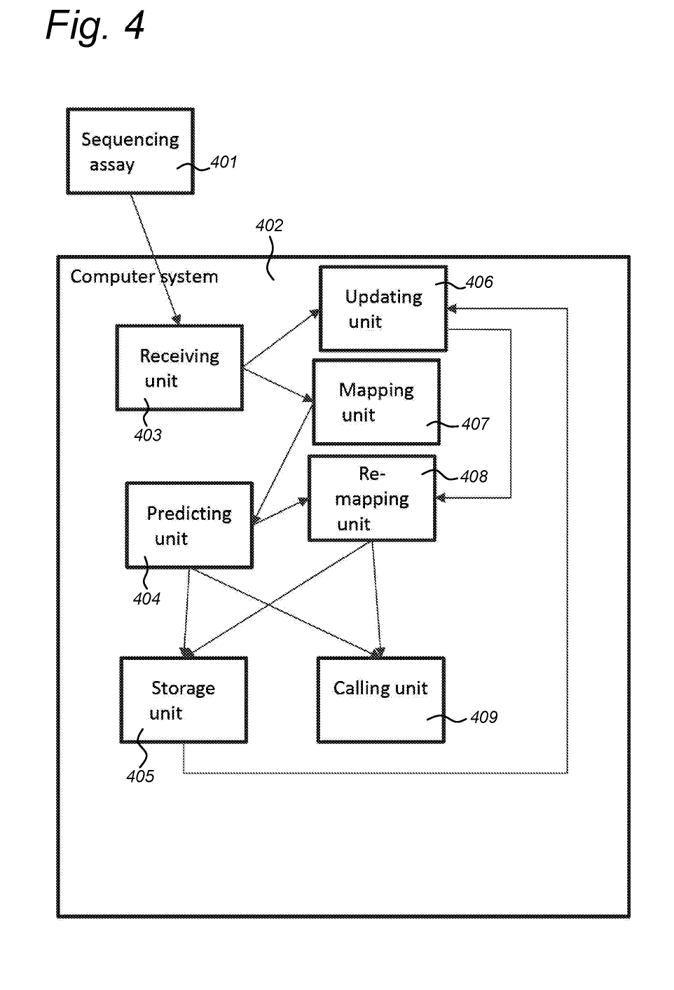

FIG. 4 shows a diagram of a sequencing system including a DNA sequencer 401 and a computer system 402, which may be a server for example. The system comprises a DNA sequencer 401 including for example an assay. The output of the DNA sequencer 401 is transmitted to computer system 402. A receiver unit 403 in the computer system 402 receives the output of the DNA sequencer 401. The receiving unit 403 forwards the output to a mapping unit 407 and an optional updating unit 406. The mapping unit 407 maps incomplete short reads to the reference DNA. The output of the mapping unit 407 is forwarded into a predicting unit 404. The predicting unit 404 predicts the missing bases and the corresponding base quality values of the incomplete short reads in the output of the sequencer 401. The output of the predicting unit 404 is forwarded to a calling unit 409 and an optional remapping unit 408. The output of the predicting unit is also forwarded to a storage unit 405. The output of the optional remapping unit 408 is forwarded to the calling unit 409 and the storage unit 405. The calling unit 409 performs variant calling, identifies a mutation in a DNA sample as compared to the reference DNA, or performs any DNA analysis algorithm. The output of the storage unit is forwarded to the optional updating unit 406. The updating unit 406 upon receiving additional information about detected base pairs of the completed sequence from the sequencing assay via the receiving unit 403, replaces the predicted base pairs and the corresponding base pair quality values of the completed sequence by the detected base pairs and the corresponding base pair quality values. The resulting output of the updating unit 406 is forwarded to the remapping unit 408. The storage unit 405 stores the data transmitted to it and retrieves data requested from it by e.g. the updating unit. Other devices or software modules may access the data stored in the storage unit 405.

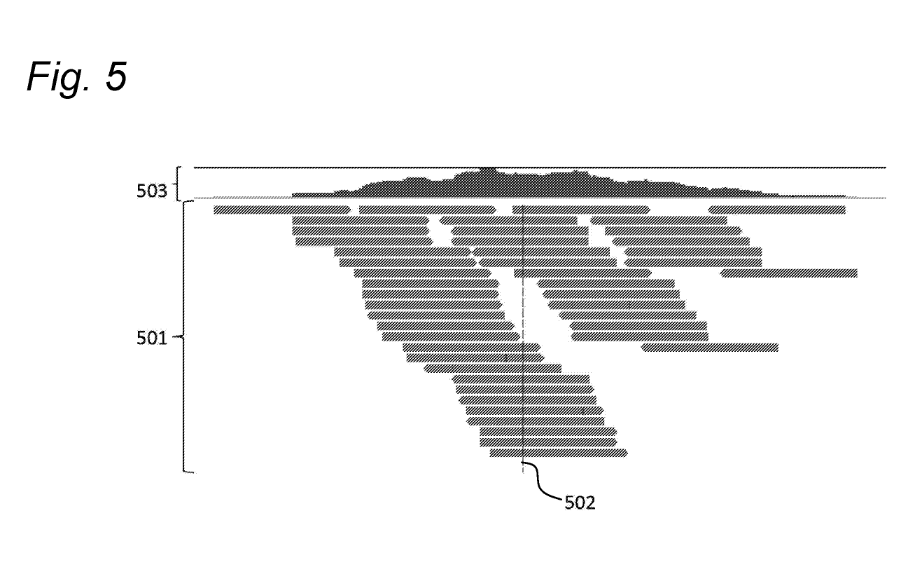

FIG. 5 shows an illustration of the way short reads oversample the DNA. In the figure, the horizontal axis represents the base pairs of a (part of a) genome. The horizontal gray bars 501 represent short reads. The vertical dashed black line 502 represents one base pair in the DNA. Clearly, one base pair occurs in a number of different short reads 501, but the position of the base pair 502 in each short read differs. Therefore, the time at which the sequencer detects that particular base pair in a short read is different for each of the short reads. As shown in FIG. 5, each base is represented (covered) by multiple short reads. Graph 503 indicates on the vertical axis the number of short reads that covers each base pair of the DNA. Therefore, if one (or multiple) of the short reads 501 is incomplete, the method can complete it using the already completed short reads that already cover the missing base pair.

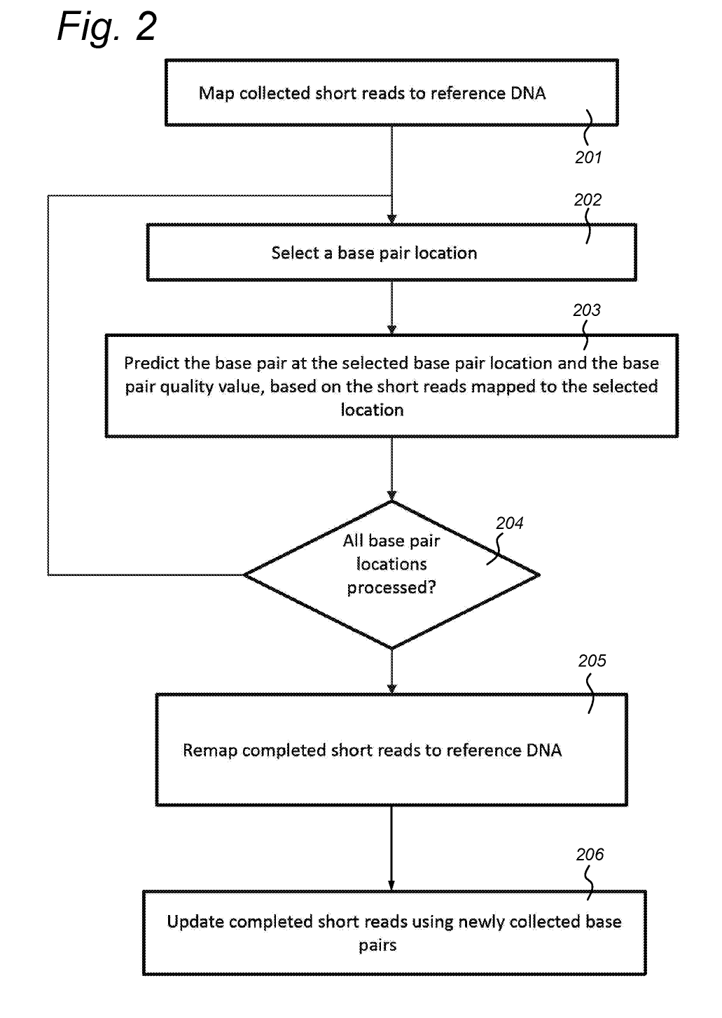

An example implementation of the process of step 102 is illustrated in FIG. 2. In step 201, the incomplete short reads are mapped to the reference DNA. Not all short reads need to be incomplete. Instead, it is possible that some of the short reads are already complete, whereas some other short reads are not yet complete. The mapping of an incomplete short read to the reference DNA may be performed in a similar way as the mapping of complete short reads is performed, by matching a pattern of base pairs of the incomplete short sequence to a corresponding pattern of base pairs on the reference DNA.

Next, for every base pair location, the available base-pair information in the collected short reads is used to predict the correct base pair character at that location. In step 202, a base pair location is selected that will be predicted. The order in which the locations are selected may be varied. In a simple example, the base pair locations are selected in order of their appearance in the reference DNA. In step 203, the base pair at the selected location is determined. An algorithm may be used that can handle sequencer errors in some base pairs. Also, base-pair quality values, which may be generated by the sequencer for each generated base pair, may be taken into account in the prediction of the correct base pair at a location. The algorithm used to perform this prediction of the correct base pair may be based on majority checking or highest base-pair quality, for example. Other algorithms to perform a prediction based on the available base pair information can also be used. Similarly, the base pair quality value of the missing bases can be predicted using the base pair quality values of the read being completed and by applying a statistical method. The predicted base pair and the corresponding base pair quality value may be stored in a preliminary DNA sequence. The predicted base pair, and optionally the corresponding base pair quality value, may also be stored in association with the corresponding base pair location in each of the short sequences that overlap the selected location.

For predicting the missing base pairs, several prediction methods can be used. In a first method, the missing base pairs may be predicted by taking a majority vote of all the base pairs from other reads that are overlapping the missing base pair in the incomplete read. In a second method, the missing base pairs of the incomplete read may be predicted as the base pairs of the overlapping read which is matching most closely to the incomplete read. In a third method the missing base pairs may be predicted by taking a weighted majority vote of all the base pairs from other reads that are overlapping the missing base pair in the incomplete read. The reads which are matching more closely to the incomplete read are given higher weightage as compared to the reads which are matching less to the incomplete read.

For predicting the missing base quality values, several prediction methods can be used. In a first method, the missing base quality values may be predicted by taking a maximum (or average) of all the base quality values from other reads that are overlapping the missing base pair in the incomplete read. In a second method, the missing base quality values can be predicted using the quality values of the bases already sequenced in the incomplete read. This can be done using a mirror image completion algorithm, where the quality value of the last base pair is predicted as equal to the quality value of the first base pair of the incomplete read; the quality value of the second to last base pair is made equal to the quality value of the second base pair; etc. In a third method, the average base quality value of a previous sequencing run of the DNA sequencing machine can be used to predict the quality values of the missing base pairs.

In step 204 it is determined whether all relevant base pair locations have been processed by step 203. For example, the aim may be to map a certain portion of the DNA; in that case it may be determined if all locations of that portion of DNA have been predicted. Alternatively, it may be chosen to process all locations of the reference DNA. If not yet all relevant base pair locations have been processed, the next base pair location is selected in step 202 and predicted in step 203.

If it is determined in step 204 that all relevant base pair locations have been processed, the next (optional) step 205 is to remap the completed short reads to the reference DNA. The remapping on the basis of completed short reads may lead to a higher accuracy of the mapping of the completed short reads. The mapping step 201 and the remapping step 205 may be performed using a similar algorithm; however, the mapping step 201 operates on the incomplete short reads, whereas the remapping step 205 operates on the completed short reads. In certain embodiments, the remapping step 205 may be identical to a mapping of a conventional dataset containing complete sequenced short reads. In step 206, as the remaining portion (missing base pairs) of the short reads is being sequenced and streamed to the computer, the predicted base pairs and the corresponding base pair quality values may optionally be replaced by the newly received base pairs and their corresponding base pair quality values. Also, optionally, the prediction of the still missing base pairs and corresponding base pair quality values may be repeated or improved using the additionally received base pairs and the corresponding base pair quality values, so that the accuracy of the predicted base pairs and the corresponding base pair quality values may be improved. Also, the mapping steps may be repeated as more base pairs and the corresponding base pair quality values of the short sequences are determined by the sequencer.

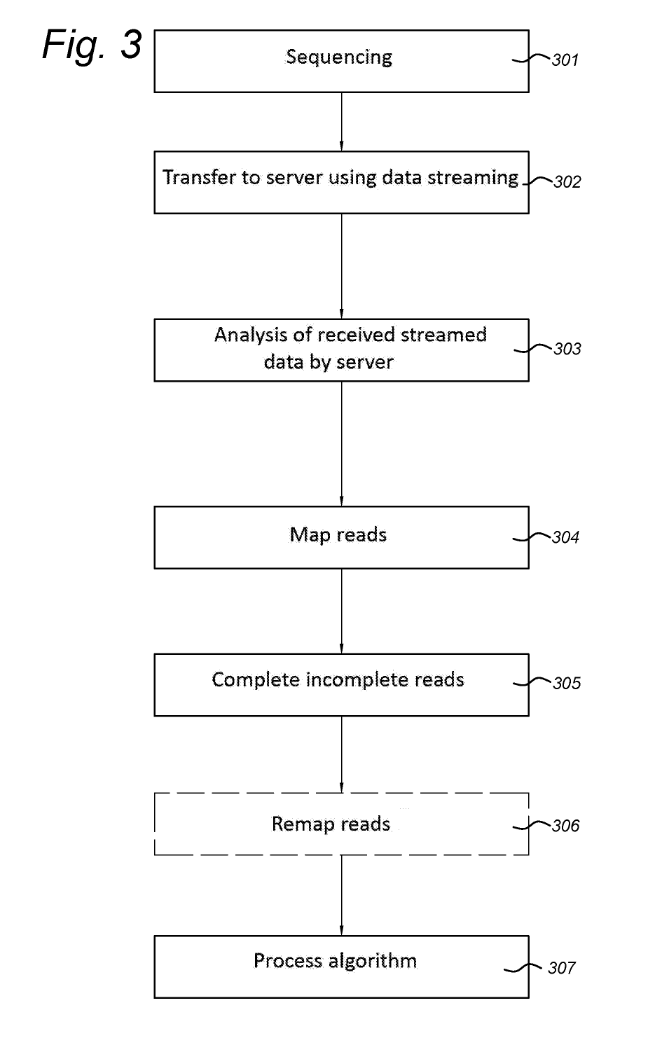

FIG. 3 illustrates a process flow according to an aspect of the present disclosure. In step 301, a DNA sequencer sequences a sample and generates base pairs and the corresponding base pair quality values associated with short sequences from the sample. In step 302, the data representing the generated base pairs and the corresponding base pair quality value is transferred to a computer server using, for example, a suitable data streaming protocol. Preferably, the data is streamed to the server as soon as possible as it becomes available by the sequencer. That is, while the sequencer is still generating further base pairs associated with the short reads, the data about the already generated base pairs is already streamed to the server.

During the streaming process 302, the server collects more and more information about the base pairs occurring in the short sequences. During this process, the server may already start its analysis process in step 303. Step 303 analyzes the data to determine whether enough bases are accumulated to start step 304.

After the analysis process of step 303, the short reads are mapped, using the information about the base pairs that have already been sequenced. Various short read mapping algorithms can be used. In some embodiments, a seed-and-extend approach may be used. In this method, substrings of the short DNA reads (known as seeds) are found which are exactly (or nearly exactly) matching in the reference DNA at one or more than one places. Then each of the location(s) of a seed in the reference DNA are visited one by one and the whole read is mapped around the location of the seed using Smith-Waterman or similar algorithms. Smith-Waterman or similar algorithms generate a mapping score. This score can be used to determine the final mapping position of the read in the reference DNA.

From the mapping step 304, the overlap of the short reads is determined. Thus, in step 305, incomplete reads are supplemented using the information from the other short sequences. The base pairs missing in one short sequence can be looked up in one or more of the other short sequences. In case base pairs have been determined for a particular location in multiple overlapping short sequences, a selection process can be performed, as explained hereinabove, to determine the most likely base pair for that location. The result of step 305 is a set of “completed” reads; that is, the information about the base pairs in each read is completed as much as possible using the available information of the other reads. The missing base pair quality values are also generated using the base pair quality values of the read being completed and by applying statistical methods.

After the supplementing step 305, the resulting supplemented short sequences may optionally be re-mapped. If the re-mapping step is skipped, the process jumps from step 305 directly to step 307. Otherwise, in step 306, the completed short reads are remapped with respect to the reference DNA. In this step, a conventional mapping procedure of short sequences may be applied to the completed short sequences to better map them with the reference DNA.

In step 307, after the incomplete reads have been completed in step 305 and optionally remapped in step 306, the completed short reads may be processed in an algorithm (e.g. a variant calling algorithm, a mutant detection algorithm, DNA analysis algorithm, etc.) as if they were regular short reads generated by the sequencer.

FIG. 4 illustrates a system for sequence analysis. The system comprises a sequencer or sequencing assay 401 for sequencing a plurality of short reads based on a sample and streaming the plurality of short reads as they are being sampled from the sequencing assay to a computer system 402. The computer system 402 may be any computer system, such as a server system or a standalone computer, or a device that is integrated with the sequencing assay 401. The computer system 402 may comprise a receiving unit 403 for receiving a plurality of sequencing reads while the sequencing assay is in progress. Herein, at least some of the sequencing reads are incomplete sequencing reads, wherein the incomplete sequencing reads are sequencing reads for which only a part of the base pairs of the sequencing read have been determined by the sequencing assay, and the missing base pairs are still in the process of being determined by the sequencing assay. The computer system may further comprise a mapping unit 407 for mapping the plurality of sequencing reads to a reference sequence. The computer system may further comprise a predicting unit 404 for predicting at least some of the missing base pairs and the corresponding base pair quality values of an incomplete sequencing read based on available base pair information of the same and other sequencing reads and applying the predicted base pairs and the corresponding base pair quality values to the incomplete sequencing read to obtain a completed sequencing read. The computer system 402 may further comprise a storage unit 405, for storing data and/or computer programs. The storage unit 405 may comprise an intangible storage media and/or a tangible storage media. The storage unit 405 may be configured to store the completed sequencing reads output by the remapping unit 408 or the predicting unit 404, for example. Also, the storage unit 405 may be configured to store the reference sequence, for example a reference DNA.

The computer system 402 may further comprise a re-mapping unit 408 for re-mapping the complete sequencing reads to the reference sequence. The computer system 402 may further comprise an updating unit 406 for, upon receiving additional information about detected base pairs of the completed sequence from the sequencing assay via the receiving unit 403, replacing the predicted base pairs and the corresponding base pair quality values of the completed sequence by the detected base pairs and the corresponding base pair quality values. The computer system may further comprise a calling unit 409 for performing variant calling, identifying a mutation in a DNA sample as compared to the reference DNA, or performing any DNA analysis algorithm, based on the completed sequencing reads. Such variant calling or identification of a mutation may be performed, for example, using a method that is known in the art by itself. The completed sequencing reads may then be treated, for example, as regular sequencing reads (e.g. short sequences), as sequencing reads that are generated by the sequencing assay entirely.

FIG. 5 illustrates that short reads may describe overlapping portions of a genome. By combining the information from overlapping short reads, the short reads may be completed or the genome may be reconstructed. The horizontal axis represents positions along a reference DNA, and the horizontal bars represent the short reads, which may be incompletely determined according to the present disclosure. In such a case, the missing base pairs may be predicted using the collected information from the overlapping other short reads.

Some or all aspects of the invention may be suitable for being implemented in form of software, in particular a computer program product. In particular the modules of the computer system and the processing steps performed by the computer system may be suitable therefor. The computer program product may comprise a computer program stored on a non-transitory computer-readable media. Also, the computer program may be represented by a signal, such as an optic signal or an electro-magnetic signal, carried by a transmission medium such as an optic fiber cable or the air. The computer program may partly or entirely have the form of source code, object code, or pseudo code, suitable for being executed by a computer system. For example, the code may be executable by one or more processors.

The examples and embodiments described herein serve to illustrate rather than limit the invention. The person skilled in the art will be able to design alternative embodiments without departing from the spirit and scope of the present disclosure, as defined by the appended claims and their equivalents. Reference signs placed in parentheses in the claims shall not be interpreted to limit the scope of the claims. Items described as separate entities in the claims or the description may be implemented as a single hardware or software item combining the features of the items described.

The present invention has been described above with reference to a number of exemplary embodiments as shown in the drawings. Modifications and alternative implementations of some parts or elements are possible, and are included in the scope of protection as defined in the appended claims.

1. A method of analyzing sequencing data associated with a sample, the method comprising:
receiving (101) by a computer system a plurality of sequencing reads while a sequencing assay is in progress of sequencing the sequencing reads, wherein at least some of the sequencing reads are incomplete sequencing reads, wherein the incomplete sequencing reads are sequencing reads for which only a part of the base pairs of the sequencing read have been determined by the sequencing assay, and the missing base pairs are still in the process of being determined by the sequencing assay;
mapping (201) by the computer system the plurality of sequencing reads to a reference sequence; and
predicting (203) at least some of the missing base pairs of an incomplete sequencing read and base pair quality values corresponding to the predicted base pairs, based on available base pair information of the sequencing reads and applying the predicted base pairs and the base pair quality values corresponding to the predicted base pairs to the incomplete sequencing read to obtain a completed sequencing read.

2. The method of claim 1, further comprising re-mapping (205) the completed sequencing reads to the reference sequence.

3. The method of claim 1, further comprising
upon receiving additional information about detected base pairs of the completed sequence from the sequencing assay, replacing (206) by the computer system the predicted base pairs and the base pair quality values corresponding to the predicted base pairs of the completed sequence by the detected base pairs and base pair quality values corresponding to the detected base pairs.

4. The method of claim 1, further comprising
performing by the computer system variant calling, identifying a mutation in a DNA sample as compared to the reference DNA, or performing a DNA analysis algorithm by analyzing (103) the completed sequencing reads.

5. The method of claim 1, further comprising
sequencing a plurality of short reads based on a sample using a sequencing assay; and
streaming the plurality of short reads as they are being sampled from the sequencing assay to the computer system.

6. A system for sequence analysis, comprising
a sequencing assay (401) for sequencing a plurality of short reads based on a sample and streaming the plurality of short reads as they are being sampled from the sequencing assay to a computer system (402); and
the computer system (402) comprising:
a receiving unit (403) for receiving the plurality of sequencing reads while the sequencing assay is in progress, wherein at least some of the sequencing reads are incomplete sequencing reads, wherein the incomplete sequencing reads are sequencing reads for which only a part of the base pairs of the sequencing read have been determined by the sequencing assay, and the missing base pairs are still in the process of being determined by the sequencing assay;
a mapping unit (407) for mapping the plurality of sequencing reads to a reference sequence; and
a predicting unit (404) for predicting at least some of the missing base pairs of an incomplete sequencing read and base pair quality values corresponding to the predicted base pairs, based on available base pair information of the sequencing reads and applying the predicted base pairs and the corresponding base pair quality values to the incomplete sequencing read to obtain a completed sequencing read.

7. The system of claim 6, wherein the computer system (402) further comprises a re-mapping unit (408) for re-mapping the complete sequencing reads to the reference sequence.

8. The system of claim 6, wherein the computer system (402) further comprises an updating unit (406) for, upon receiving additional information about detected base pairs of the completed sequence from the sequencing assay, replacing the predicted base pairs and the base pair quality values corresponding to the predicted base pairs of the completed sequence by the detected base pairs and base pair quality values corresponding to the detected base pairs.

9. The system of claim 6, wherein the computer system (402) further comprises an analysis unit (409) for performing variant calling, identifying a mutation in a DNA sample as compared to the reference DNA, or performing a DNA analysis algorithm based on the completed sequencing reads.

10. A computer program product comprising instructions for causing a computer system to perform the method according to claim 1.
